SEQUENCE LISTING (22 sequences)

SEQ ID NO: 1 - DNA, 13 bp, Artificial Sequence
  PNA probe 180w
agccaggngaaac

SEQ ID NO: 2 - DNA, 14 bp, Artificial Sequence
  PNA probe 180wt
tgagccatgataac

SEQ ID NO: 3 - DNA, 14 bp, Artificial Sequence
  PNA probe 180m
tgagccaagataac

SEQ ID NO: 4 - DNA, 17 bp, Artificial Sequence
  PNA probe 180n
cagagccacgagatacg

SEQ ID NO: 5 - DNA, 15 bp, Artificial Sequence
  PNA probe 204vw
tcatccatataactg

SEQ ID NO: 6 - DNA, 13 bp, Artificial Sequence
  PNA probe 204v
catccacataact

SEQ ID NO: 7 - DNA, 15 bp, Artificial Sequence
  PNA probe 204vn
tcatccaaataactg

SEQ ID NO: 8 - DNA, 15 bp, Artificial Sequence
  PNA probe 204vn
tcatccagataactg

SEQ ID NO: 9 - DNA, 14 bp, Artificial Sequence
  PNA probe 204iw
atcatccatataac

SEQ ID NO: 10 - DNA, 17 bp, Artificial Sequence
  PNA probe 204il
catcatctatataactg

SEQ ID NO: 11 - DNA, 17 bp, Artificial Sequence
  PNA probe 204i2
catcatcgatataactg

SEQ ID NO: 12 - DNA, 15 bp, Artificial Sequence
  PNA probe 204i2
catcatcgatataac

SEQ ID NO: 13 - DNA, 16 bp, Artificial Sequence
  PNA probe 204i3
catcatcaatattaac

SEQ ID NO: 14 - DNA, 15 bp, Artificial Sequence
  PNA probe 204in
catcatctcatataa

SEQ ID NO: 15 - DNA, 13 bp, Artificial Sequence
  PNA probe 207w
aataccacatcat

SEQ ID NO: 16 - DNA, 15 bp, Artificial Sequence
  PNA probe 207i1
caatacaatatcatc

SEQ ID NO: 17 - DNA, 15 bp, Artificial Sequence
  PNA probe 207i2
caatactatatcatc

SEQ ID NO: 18 - DNA, 15 bp, Artificial Sequence
  PNA probe 207n
caatacgaaatcatc

SEQ ID NO: 19 - DNA, 20 bp, Artificial Sequence
  Primer BF105
tcctgctgctatgcctcatc

SEQ ID NO: 20 - DNA, 31 bp, Artificial Sequence
  Primer BF112
ttccgtcgacatatcccatgaagttaaggga

SEQ ID NO: 21 - DNA, 25 bp, Artificial Sequence
  Primer HB-F3
cttgtattcccatcatcccatcatc

SEQ ID NO: 22 - DNA, 25 bp, Artificial Sequence
  Primer HB-R2
gaaaagaaaattggtaacagcggta

(19) United States
(12) Patent Application Publication
(10) Pub. No.: US 2008/0233557 A1
(43) Pub. Date: Sep. 25, 2008

(54) PNA Probes, Kits, and Methods for Detecting Lamivudine-Resistant Hepatitis B Viruses

(75) Inventors: Hee Kyung PARK, Daejeon (KR); Hyunil LEE, Daejeon (KR); Jae Jin CHOI, Daejeon (KR); Serka KIM, Gyeonggi-do (KR)

Correspondence Address: THE WEBB LAW FIRM, P.C., 700 KOPPERS BUILDING, 436 SEVENTH AVENUE, PITTSBURGH, PA, 15219, US

(73) Assignee: PANAGENE INC., Daejeon (KR)

(21) Appl. No.: 11/860,983

(22) Filed: Sep. 25, 2007

(30) Foreign Application Priority Data
Mar. 23, 2007 (KR) 10-2007-0028684

(51) Int. Cl. C12Q 1/70; C07H 21/04

(52) U.S. Cl. 435/5; 536/24.32

(57) ABSTRACT

Disclosed are peptide nucleic acid (PNA) probes to detect lamivudine resistant mutants of hepatitis B virus (HBV), which causes acute and chronic hepatitis, kits for detecting lamivudine resistant HBV comprising the PNA probes, and methods for detecting lamivudine resistant HBV by using the kits. They can accurately detect mutations of rtL180M, rtM204V, rtM204I and rtV2071 within B and C domains of HBV DNA polymerase gene, the main cause of lamivudine resistance, as well as mixed mutants of more than one mutant. They can detect lamivudine resistant HBV with high specificity and sensitivity.

### FIELD OF THE INVENTION

The present invention relates to detection of antibiotic resistant point mutations (mutants) of hepatitis B virus (hereinafter, referred to as ‘HBV’) which causes acute and chronic hepatitis, by using PNA probes. More specifically, the invention relates to PNA probes for detecting point mutations in HBV DNA polymerase gene associated with resistance to lamivudine, a therapeutic agent for chronic hepatitis B, kits for detecting lamivudine resistant HBV comprising the probes, and methods for detecting lamivudine resistant HBV using the kits.

### BACKGROUND OF THE RELATED ART

HBV is a semicircular double stranded DNA virus consisting of pre-core/core, pre-s/s, P and X genes, four open reading frames of about 3.2 kb, which causes acute or chronic hepatitis [Management of viral Hepatitis B, Chutima Pramoolsinsup, 2002, J Gastroenterology and Hepatology Castrol, S125-S145]. In spite of development of hepatitis B vaccines and therapies, about 300,000,000 individuals are infected with hepatitis B all over the world. In 3,000,000 individuals among them, hepatitis B is converted to chronic liver diseases such as hepatic cirrhosis and liver cancer to become one of major causes of deaths of adults [Hepatitis B: global importance and need for control, Maynard J E, Vaccine, 1990, 8:18-28].

A variety of researches have been, performed for the therapy of chronic hepatitis B, including on immunomodulatory agents and antiviral agents. Among them, lamivudine ((−)-B-L-2′,3′-dideoxy-3′-thiacytidine, 3T) is an effective therapeutic agent as an nucleoside analogue for inhibiting proliferation of HBV by the mechanism of reducing the activity of RNA-dependent DNA polymerase (reverse transcriptase) of HBV, and widely employed as primary therapeutic agent for chronic active hepatitis B at present [Effects of extended lamivudine therapy in Asian patients with hepatitis D, Liaw et al., 2000, J Gastroenterology, 119:172-180; A one year trial of lamivudine for chronic hepatitis B, Lai et al., N Engl J Med 1998:339 L61-38]. Lamivudine has been known as an effective therapeutic agent because it can be administered orally, shows excellent histological, biological and biochemical improvements after treatment, and is safe. However, lamivudine resistant viruses appear in 10˜15% of patients after administration for two years or more, and at least 50% of patients after administration for three years or more [Prevalence and clinical correlates of YMDD variants during lamivudine therapy for patients with chronic hepatitis B, Lai et al., 2003, J Clin infect Dis, 36:687-696; Evaluation of wild type and mutants of the YMDD motif of hepatitis B virus polymerase during lamivudine therapy, Xinxin Zhang et al., 2003, J Gastroenterology and Hepatology, 18:1353-1357]. It has been known that lamivudine resistance occurs from mutation at YMDD motif (tyrosine-methionine-aspartate-aspartate amino acid motif) of HBV polymerase (reverse transcriptase) gene. The YMDD motif is a site to which lamivudine binds, wherein mutation rarely occurs in general. But after prolonged treatment with lamivudine, methionine (M) at codon 552 is varied to isoleucine (I), valine (V) or serine (S), and leucine (L) at codon 528 to methionine, in YMDD motif, to inhibit the binding of lamivudine, thereby causing resistance to lamivudine [Lamivudine resistance in hepatitis B: mechanisms and clinical implications, Fischer et al., 2001, Drug Resistance Updates, 4:118-128]. Lamivudine resistant mutations were previously designated according to different numbering systems depending on seven genotypes of HBV, but can be now designated by the standardized nomenclature regardless of the genotypes according to the rt domain numbering system which numbers from the starting point of each domain, suggested by Stuyver in 2001 [Nomenclature for antiviral resistant human hepatitis B virus mutations in the polymerase region, Stuyver et al., 2001, Hepatology 33:751-757]. The mutations designated according to the standardized nomenclature are rtL80V/I in domain A, rtL180M in domain B, and rtM204I, rtM204V, rtM204S and rtV207I in domain C of HBV DNA polymerase gene (see FIG. 1). Among them, rtL180M corresponds to variation of amino acid 528, 526, 515 or 525, and rtM204V/I to variation of amino acid 552, 550, 539 or 549, as previously reported.

These HBV variants are lamivudine resistant, that is, lamivudine cannot inhibit the proliferation of HBV. Thus, effective therapy can be only made by administering other agents such as adefovir and famciclovir, or by suppressing the generation of HBV variants by means of combination therapy of two agents from the early stage of therapy [Suppressing hepatitis B without resistance-so far, so good, Mailliard et al., 2006 N Engl J Med, 348:848-850]. Since early diagnosis of lamivudine resistant HBV variants in a patient with chronic hepatitis B is very important for treatment and planning of remedy of the patient, a method is required to sensitively and rapidly detect the presence of lamivudine resistance.

As a method to detect lamivudine resistant HBV mutants, nucleotide sequencing or pyrosequencing which has been recently studied is employed. However, these methods require expensive equipments, and may not be able to detect the variants which occupy not more than 20% of total viruses at the early stage of developing the resistance [Journal of the Korean Society for Laboratory Medicine (KSLM), Vol. 23, No. 4, 2003; Pyrosequencing for Detection of Lamivudine-Resistance Hepatitis B virus, Anna et al., 2004, J Clin Microbiol., 4788-4795].

In the meanwhile, a method of detecting mutations by using PCR (polymerase chain reaction) requires a number of amplification reactions because it has to use individual primers for various mutations [Detection of YMDD mutation using mutant-specific primers in chronic hepatitis B patients before and after lamivudine treatment, Cha-Ze et al., 2006, World Gastroenterol, 12(33):5301-5305]. A method of detecting mutations by using RFLP (Restriction fragment length polymorphism) cannot detect the transfer stage to variation, and has a low reproducibility [Two sensitive PCR-based methods for detection of hepatitis B virus variants associated with reduced susceptibility to lamivudine, Allen et al., 1999, J Clin Microbiol, 37:3338-3347]. As an alternative method, line probe assay (LIPA) can be employed, but this method has drawbacks that false positive may occur by the mutation of adjacent sites, and that only one sample can be tested in one strip, but a number of samples cannot be detected simultaneously [Monitoring drug resistance in chronic hepatitis B virus-infected patients during lamivudine therapy: evaluation of performance of INNO-Lipa HBV DR assay, Lok A S et al., 2002, J Clin Microbiol, 40:3729-3734].

Recently, a method to detect lamivudine resistant HBV variants by using DNA microarray (DNA chip) technique has been developed [Korean Patent Laid-Open No. 2005-0015407; Oligonucleotide chip for Detection of Lamivudine-resistant Hepatitis B virus, Jang et al., J Clin Microbiol 2004, 4181-4188]. In particular, Korean Patent Laid-Open No. 2005-0015407 discloses a microarray comprising target probes for detection of drug resistant HBV; QC probes for quality control during hybridization; and, negative control probes for determination of presence and ratio of one or more mixed mutant type(s) with wild types, measurement of background due to nonspecific cross hybridization and discrimination of homozygotes and heterozygotes. It also discloses simultaneous detection of HBV, quality control of the microarray and determination of presence and ratio of mixed mutant types, and determination of positive and false positive for each probe. The above patent mentions that DNA analogues such as PNA (peptide nucleic acids), LNA (locked nucleic acid) and HNA (hexitol nucleic acid) may be usable for the microarray, but specifically discloses only DNA chips and does not disclose any chip using the DNA analogue. The DNA chip can rapidly detect drug resistant point mutations of HBV in a short time. In addition, as they comprise negative control probes, the DNA chips can be a very sensitive means which can detect mixed mutant types with wild types, and thus, they are widely utilized for diagnosis of drug resistance.

Further, methods for detecting variants and genotypes thereof by means of microsphere suspension arrays are recently employed. According to the methods, various biological substances such as DNA, antigens, antibodies, enzymes, substrates and receptors can be attached as probes for detecting a target substance to beads made of polystyrene. The target substance is hybridized with individual beads to which the probes have been attached, and the beads flowing through fast flowing fluid are detected by using lasers of two types. One laser detects fluorescence of beads wherein hybridization with the target substance occurred, while the other laser detects and selects identification numbers attached to the beads, to detect variations. Thus, a sample of smaller amount can be analyzed as compared to common DNA chip which immobilizes the probes on a glass slide or the like. Due to high sensitivity, a large number of samples can be treated in a short time. In addition, the method is very useful, since the variation can be detected with high specificity and discrimination [Suspension array technology: evolution of the flat-array paradigm, Facile, John P. Nolan and Larry A. Sklar, 2002, Trends in Biotechnology, vol. 20; Microsphere suspension array technology for SNP detection in cattle, Dunbar et al., 2003, Engineering in Medicine and Biology magazine, 22: 158-162].

However, the DNA chip or microsphere suspension array has problems of denaturation of DNA or decrease in reactivity over storage time, because the immobilized DNA probes themselves have very low biological and chemical stability to nucleases or the like (See Korean Patent Laid-Open No. 2006-0091708).

In order to overcome the instability of DNA itself, various DNA analogues have been developed. Among them, PNA (peptide nucleic acid) has beer developed by Nielsen in 1991. As shown in FIG. 2, in PNA, phosphodiester bonds of DNA are replaced by peptide bonds. Since PNA has adenine, thymine, guanine and cytosine like DNA, it can perform base-specific hybridization with DNA or RNA. In particular, its backbone structure with peptide bonds alters anionic property of phosphate backbone of DNA or RNA to neutral. The removal of electrostatic repulsion between anions resulting from neutralization of anionic property directly contributes to the increase in binding ability upon hybridization. As a result, it has increased hybridization rate and specificity, and thus, has improved S/N (signal to noise) ratio. In addition, PNA is more stable than DNA or RNA because biological degrading enzymes such as nucleases cannot recognize PNA [See PNA, sequence-selective recognition of DNA by strand displacement with a thymine-substituted polyamide, P. E. Nielsen et al., 1991, Science, 254, 1497-1500].

As described above, PNA, which has high hybridization ability and stability while retaining the functions of DNA or RNA, is recognized as a promising alternative to DNA that can complement drawbacks of DNA. Thus, extensive studies have been conducted for analysis or diagnosis with PNA oligomers in place of DNA oligomers [See Korean Patent Laid-Open Nos. 2006-0091708 and 2005-0122544; Peptide nucleic acids on microarrays and other biosensors, Brandt O and Hoheisel J D, 2004, Trends in Biotechnology, 22, 617-622; Detection of target DNA using fluorescent cationic polymer and peptide nucleic acid probes on solid support, Frederic R Raymond et al., 2005, BMC technology, 5, 1-5].

### SUMMARY OF THE INVENTION

In order to solve the problems of the prior arts as described above, the present inventors manufactured PNA probes (by using PNA having the advantages as mentioned above) which can detect single or mixed mutants of lamivudine resistant HBV, and manufactured PNA chips by employing the PNA probes. They confirmed that the point mutation of lamivudine resistant HBV can be detected with high specificity and sensitivity by using them, and completed the present invention.

An object of the present invention is to provide PNA probes which can detect lamivudine resistant HBV with high specificity and sensitivity, while being stable to biological enzymes.

Another object of the invention is to provide a kit for detecting lamivudine resistant HBV, which comprises said probes.

Still another object of the invention is to provide a method for detecting lamivudine resistant HBV by using said detection kit.

One aspect of the present invention relates to a PNA probe capable of specifically binding with wild type or mutant type at codon 180 in domain B, or codon 204 or 207 in domain C of HBV DNA polymerase gene associated with lamivudine resistance, which consists of the nucleotide sequence as set forth in any one of SEQ ID Nos. 1, 2, 3, 5, 6, 9, 10, 11, 12, 13, 15, 16 and 17.

A second aspect of the invention relates to a negative control PNA probe, which does not hybridize with the HBV DNA polymerase gene, and consists of the nucleotide sequence as set forth in any one of a nucleotide sequence from SEQ ID Nos. 4, 7, 8, 14 and 18.

A third aspect of the invention relates to a kit for detecting lamivudine resistant HBV, which comprises PNA probes and a support, and optionally, negative control probes, the PNA probes and the negative control probes being immobilized on the support, wherein the PNA probes are capable of specifically binding with wild type or mutant type at codon 180 in domain B, or codon 204 or 207 in domain C of HBV DNA polymerase gene associated with lamivudine resistance, and the negative control probes have varied nucleotide sequences from those of the PNA probes so as not to hybridize with the HBV DNA polymerase gene.

A fourth aspect of the invention relates to a method for detecting lamivudine resistant HBV, which comprises the steps of:

(a) adding a reaction sample containing a target DNA to the detection kit;

(b) subjecting the PNA probes and the target DNA to hybridization;

(c) detecting the signal resulting from the hybridization of PNA and DNA.

### BRIEF DESCRIPTION OF DRAWINGS

FIG. 1 illustrates domains and nomenclature of mutation of HBV DNA polymerase gene;

FIG. 2 compares the basic structures of PNA and DNA;

FIG. 3 is a photograph showing the results of electrophoresis on 2% agarose gel for the nucleic acids of various lamivudine resistant HBV mutants amplified with the primers shown in Table 2;

FIG. 4 is a schematic diagram showing a chip for detecting 4 samples in a slide (a), spot positions of the chip (b), and types of probes in the spot positions (c), according to one embodiment of the invention;

FIGS. 5a to 5l are photographs showing the results of detecting individual wild types and mutations associated with lamivudine resistance of HBV by using the PNA chip according to one embodiment of the present invention;

FIG. 6 is a set of graphs showing the quantified detection signals of lamivudine resistant HBV mutations on the conventional DNA chip and the PNA chip according to the invention;

FIG. 7 is a set of graphs comparatively showing the specific signals and S/N ratios of individual probes of the conventional DNA chip and the PNA chip according to the present invention.

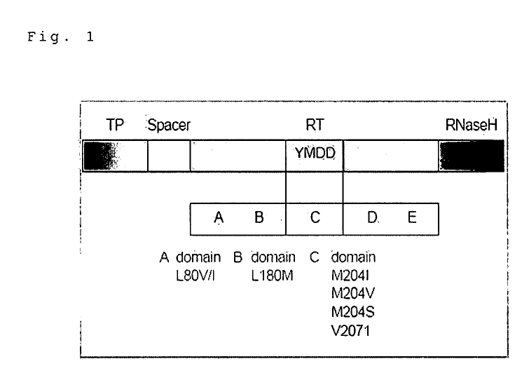

Other and further objects, features and advantages of the invention will appear more fully from the following description.

### DETAILED DESCRIPTION OF THE PREFERRED EMBODIMENT

Throughout the specification, the terms “mutation”, “variation” and “variant” are interchangeably used with same meaning. HBV “DNA polymerase” and “reverse transcriptase” are also interchangeably used with same meaning.

PNA probes for detecting lamivudine resistant HBV, and a kit and a method for detecting lamivudine resistant HBV have been completed according to the following procedures.

1. Obtainment of Clone and Sequence Analysis

DNA Samples from patients suffered from chronic hepatitis B with HBV infection under lamivudine therapy were PCR amplified by means of the primers shown in Table 2, and the amplified PCR product was cloned to a plasmid vector. The clone thus obtained was transformed to E. coli JM109 to obtain a large amount of DNA. The clonal DNA thus obtained was sequenced to confirm the genotypes of wild type and mutant type of gene associated with lamivudine resistance of HBV. The clone of which the genotype was confirmed by sequencing was used as a standard or control sample for the establishment of the reaction conditions of the PNA chip of the invention. The clinical samples of which the genotypes of wild type and mutant type of HBV associated with lamivudine resistance had been confirmed were used for the analysis of accuracy of the PNA chip according to the present invention.

2. Design and Manufacture of PNA Probe

As target probes for detecting lamivudine resistant HBV, PNA probes were designed to comprise nucleotide sequences that can specifically bind with the target gene associated with lamivudine resistance, i.e. nucleotide sequences of point mutations at rtM-204V wherein methionine (M) at codon 204 is replaced by valine (V), rtM204I wherein said methionine (M) is replaced by isoleucine (I), and rtV207I wherein valine (V) at codon 207 is replaced by isoleucine (I), in YMDD motif of domain C, and rtL180M wherein leucine (L) at codon 180 is replaced by methionine (M) in domain B, of HBV DNA polymerase gene. The probe for detecting the point mutation at each site was designed to have the length of 13mer to 17mer. Exceptionally, since the site for detecting the mutation at codon 180 has the nucleotide sequence comprising 60% or more of pyrimidine bases so that the nucleotide sequence that complimentarily binds with them inevitably comprises 60% or more of purine bases, a universal base was applied as disclosed in Korean Patent Application No. 2006-0129517 filed by the Applicant, the whole contents of which are incorporated hereto by reference (SEQ ID No. 1) As described by Hemavathi [Henavathi et al., 1999, Organic Letters Vol. 1, No. 10, 1639-1641], PNA monomer comprising a universal base shown in Formula I was synthesized and employed.

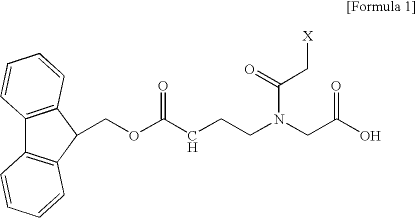

wherein a UB (universal base) is an artificial base which maximizes base stacking while maintaining DNA double helix structure. Examples of UB include purine of formula (a); 3-nitropyrrole of formula (b); 5-nitroindole of formula (c); benzimidazole compounds of formula (d); benzene and fluorobenzenes of formula (e) such as 4-fluorobenzene and pentafluorobenzene; xanthine of formula (f); pyridopyrimidine compound of formula (g); a compound containing N or CH at X, Y position of sugar chain of formula (h), such as hypoxanthine; cytosine compounds of formulas (i) to (k); adenine compounds of formula (1); 3-aminocarbonylpyrrole of formula (m); nitrodiazole of formula (n); compounds containing S or Se in the ring of formula (o); diazole compound of formula (p); triazole compound of formula (q); and β-heptafluoronaphthalene of formula (r) (See U.S. Pat. No. 5,438,131 and Kathryn et al., 2002, Chem. Commun. 2206-2207).

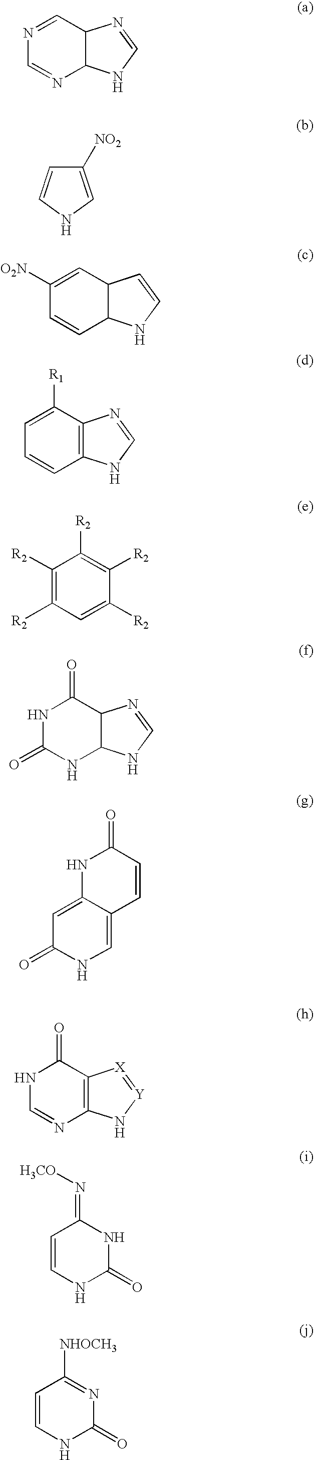

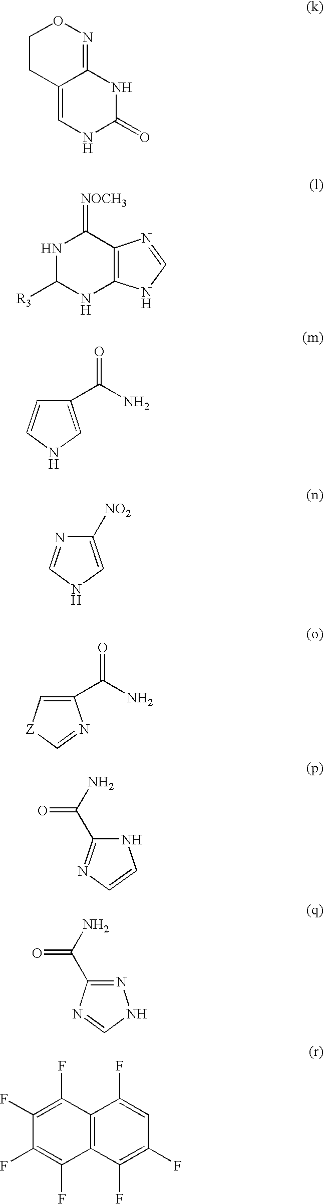

wherein

R₁ is H, NO₂ or NH₂,

R₂ is H or F,

R₃ is H or NH₂,

X and Y independently of each other represent CH or N, and

Z is S or Se.

UB is preferably 3-nitropyrrole of formula (b) or 5-nitroindole of formula (c), particularly, 3-nitroindole.

According to the present invention, negative control probes are designed to detect mutations where more than one types of mutation are mixed, and to distinguish false positive due to nonspecific cross hybridization. The negative control probes are designed by artificially varying at least one nucleotide sequence by means of substitution, insertion or deletion, on the basis of nucleotide sequences of target probes containing nucleotide sequences for detecting wild type and mutant type of each codon so that they do not hybridize with the target DNA. Table 1 shows SEQ ID Nos., nucleotide sequences, codon numbers and variations of amino acids in domains B and C in HBV polymerase gene of the PNA probes according to the present invention.

TABLE 1

| | Varia- | | | |
|---|---|---|---|---|
| Co- | tion of | Designation | | SEQ ID |
| don | amino acid | of probes | Sequence (5′→3′) | No. |
| 180 | rtL1-180 | 180w | AGCCAGGNGAAAC | 1 |
| | rtL2-180 | 180wt | TGAGCCATGATAAC | 2 |
| | rtL-180M | 180m | TGAGCCAAGATAAC | 3 |
| | N-1 | 180n | CAGAGCCACGAGATACG | 4 |
| 204 | rtM1-204 | 204vw | TCATCCATATAACTG | 5 |
| | rtM-204V | 204v | CATCCACATAACT | 6 |
| | N-2 | 204vn | TCATCCAAATAACTG or | 7 or |
| | | | TCATCCAGATAACTG | 8 |
| 204 | rtM2-204 | 204iw | ATCATCCATATAAC | 9 |
| | rtM2-04il | 204il | CATCATCTATATAACTG | 10 |
| | rtM204i2 | 204i2 | CATCATCGATATAACTG | 11 or |
| | | | or CATCATCGATATAAC | 12 |
| | rtM-204i3 | 204i3 | CATCATCAATATTAAC | 13 |
| | N-3 | 204in | CATCATCTCATATAA | 14 |
| 207 | rtV-207 | 207w | AATACCACATCAT | 15 |
| | rtV-207il | 207il | CAATACAATATCATC | 16 |
| | rtV-207i2 | 207i2 | CAATACTATATCATC | 17 |
| | N-4 | 207n | CAATACGAAATCATC | 18 |

N: universal base-PNA

As can be seen from Table 1, the probes according to the present invention consist of nucleotide sequences as set forth in SEQ ID Nos. 1 to 18, respectively, depending upon the types of point mutation occurring in domain B or C of HBV reverse transcriptase, which are main mutational sites causing lamivudine resistance.

SEQ ID Nos. 1 and 3 are nucleotide sequences which complimentarily bind with genotypes CTG and TTG having the phenotype of wild type of ¹⁸³leucine; SEQ ID No. 2 is a nucleotide sequence that complimentarily binds with genotype ATG having mutant type wherein ¹⁸⁰leucine is replaced by methionine; and SEQ ID No. 4, as a negative control probe for codon 180, is a nucleotide sequence that complimentarily binds with nucleotide sequence of GTG substituted by G which belongs to neither wild type nor mutant type.

SEQ ID No. 5 is a nucleotide sequence which complimentarily binds with genotype ATG having the phenotype of wild type of ²⁰⁴-methionine; SEQ ID No. 6 is a nucleotide sequence that complimentarily binds with genotype GTG of mutant type wherein ²⁰⁴-methionine is replaced by valine; and SEQ ID No. 7, as a negative control probe for codon 204, is a nucleotide sequence that complimentarily binds with nucleotide sequence of TTG substituted by T which belongs to neither wild type nor mutant type. In this case, C may be used in place of T for substitution (SEQ ID No. 8).

SEQ ID No. 9 is a nucleotide sequence which complimentarily binds with genotype ATG having the phenotype of wild type of ²⁰⁴-methionine; SEQ ID Nos. 10, 11, 12 and 13 are nucleotide sequences that complimentarily bind with genotypes ATA, ATC and ATT, respectively, of mutant type wherein ²⁰⁴-methionine is replaced by isoleucine; and SEQ ID No. 14, as a negative control probe for codon 204, is a nucleotide sequence that has substitution and insertion so as to be hybridized with neither wild type nor mutant type.

SEQ ID No. 15 is a nucleotide sequence which complimentarily binds with genotype GTG having the phenotype of wild type of ²⁰⁷valine; SEQ ID Nos. 16 and 17 are nucleotide sequences that complimentarily bind with genotypes ATT and ATA, respectively, of mutant type wherein ²⁰⁷valine is replaced by isoleucine; and SEQ ID No. 18, as a negative control probe for codon 207, is a nucleotide sequence that complimentarily binds with nucleotide sequence of CTT substituted by C and T so as to hybridize with neither wild type nor mutant type.

The PNA probes according to the invention may have a functional group(s) required for immobilization such as amine or thiol at the N-terminus, in order to achieve efficient immobilization on a support, but the type of the functional group is not particularly limited. If the PNA probes according to the invention have an amine group at the N-terminus, they preferably contain a multi-amine linker of Formula (2) as disclosed by Korean Patent Application No. 2006-128938, the whole contents of which are incorporated hereto by reference, but the scope of the invention is not limited thereto.

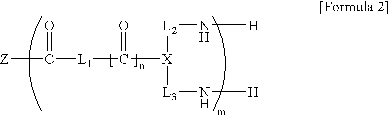

wherein

L₁, L₂ and L₃ independently of one another represent a chemical bond, or C₁˜Clo linear hydrocarbon chain, which may further comprise 1˜3 oxygen atom(s);

X is CH or N;

m is an integer from 2 to 10; and

n is 0 or 1.

The PNA oligomers employed in the present invention can be synthesized according to the method of Korean Patent Registration No. 464261, by using PNA monomer protected with Bts (benzothiazolesulfonyl) group, or PNA monomers protected with conventional Fmoc (9-fluorenylmethyloxycarbonyl) or t-Boc (t-butoxycarbonyl). [See J Org Chem 59, 5767-5773, J Peptide Sci 3, 175-183; Tetrahedron Lett 22, 6179-6194]. PNA having multi-amine linker as shown in Formula (2) is synthesized by sequentially linking dendron monomers, each having one carboxylic group and two or more branched amine groups, to the N-terminus of the synthesized PNA or of the spacer linked to the synthesized PNA, twice or more. Specifically, PNA can be synthesized through the following three steps: (i) elimination of protective groups linked to amine groups of PNA oligormers (deprotection), (ii) coupling of PNAs with dendron monomers having multi-amine linkers, and (iii) capping (See Korean Patent Application No. 2006-128938).

3. Manufacture of PNA Chip

The probes designed in the above 2. are immobilized on a support of silica, semiconductor, plastic, gold, silver, magnetic molecules or a polymeric substance such as nylon and poly(dimethylsiloxane) (PDMS), cellulose and nitrocellulose, particularly, a glass slide.

The form of the support is not particularly limited, but it may be, for example, a hand holdable thin plate such as a glass slide, a tube, or a bead having the diameter of 0.1 mm or less which can be transferred in admixture with liquid. The surface of the support can be functionalized with a functional group such as aldehyde group, carboxylic group, epoxy group, isothiocyanate group, N-hydroxysuccinimidyl group, activated ester group, particularly, with epoxy group. Upon immobilization of the probes, the functional group such as residual amine or epoxy group is blocked and treated to reduce the background signal (See Example 5).

4. Establishment of Conditions for Reaction and Analysis on PNA Chip

The method for detecting genotypes of HBV according to the present invention comprises the steps of:

(a) adding a reaction sample containing a target DNA to the kit as described above;

(b) subjecting PNA probes in the kit and the target DNA to hybridization;

(c) detecting a signal from the hybridization of PNA and DNA.

In step (a), it is preferable to use the target DNA prepared by amplifying DNA isolated from an HBV-infected patient by primary PCR with primers of SEQ ID Nos. 19 and 20, followed by amplifying the primary PCR product by secondary PCR with 5′-biotinylated primers of SEQ ID Nos. 21 and 22, as shown in Table 2.

In step (b), it is preferable to use an appropriate hybridization buffer to facilitate hybridization of the PNA probes with the target nucleic acid. It is also preferable to carry out hybridization with adding Streptavidin-cyanine 5 that binds with biotin labeled at the 5′ terminus of the primer to develop color. Upon completion of the hybridization, it is preferable to use a washing buffer that can effectively remove unreacted residual target nucleic acid and non-specific reactants.

In step (c), any detection means may be employed, including optical, electrochemical and other means that detect signals from DNA/DNA hybridization. For instance, the detection means may include cy 5, biotin linkable compound, cy3 or the like, but they are not limited thereto. It is preferable to scan fluorescence emitted from the binding of biotin labeled at the 5′ terminus of the target nucleic acid with Streptavidin-cyanine 5.

### EXAMPLES

Hereinafter, the present invention will be illustrated in more detail with reference to specific examples. However, the present invention is not limited by those examples in any manner, and it is apparent to a person having ordinary skill in the art that various alterations and modifications can be made within the spirit and scope of the invention.

### Example 1

### Synthesis of PNA Oligomer for Detecting Lamivudine Resistant HBV

Seventeen (17) PNA probes for the detection of lamivudine-resistant HBV gene were prepared to have nucleotide sequences to detect point mutations having a single nucleotide sequence difference from wild type gene associated with lamivudine resistance, as shown in Table 1. Each probe was synthesized to have a multi-amine linker at the N-terminus for immobilization on a glass slide.

1) Preparation of PNA Oligomer

According to the procedures described in Korean Patent Registration No. 464261, PNA oligomer was synthesized from PNA monomer protected with Bts (Benzothiazolesulfonyl) group and a functionalized resin by solid phase synthesis. 8-(9H-Fluoren-9-ylmethoxycarbonylamino)-3,6-dioxa-octanoic acid was introduced twice as a spacer at the N-terminus. PNA attached to the resin was employed for the subsequent reaction.

2) Preparation of PNA Probes Having Multi-Amine Linker

The PNA attached to the resin prepared from above 1) was treated with a solution of 1 M piperidine in DMF (dimethylformamide) to eliminate Fmoc protective group at the N-terminus, and then, washed with DMF three times (Stage (a)) To 1 equivalent of bis Fmoc monomer were added 1 equivalent of HOBt (1-hydroxybenzotriazole), 2 equivalents of DIC (diisopropylcarbodiimide) and DMF. After shaking for 1 hour, the mixture was washed with DMF three times (Stage (b)). DMF containing 5% acetic anhydride and 6% lutidine was added thereto, and the mixture was shaken at ambient temperature for 5 minutes, and then washed with DMF three times (Stage (c)). Stages (a) to (c) were repeated twice or three times, and finally, Stage (a) was repeated to eliminate the Fmoc protective group. For example, to introduce four (4) amine groups, the stages were repeated twice, and to introduce eight (8) amine groups, the stages were repeated three times. The resin with the attached PNA was treated with m-cresol/TFA (trifluoroacetic acid) (¼ v/v) solution for 2 hours to detach PNA from the resin. Precipitation with ether and purification by HPLC gave the probes having multi-amine linkers of SEQ ID Nos. 1 to 7 and 9 to 18 (in SEQ ID No. 1, N is 3-nitroindole-PNA).

### Example 2

### Synthesis of Primers for Preparing Lamivudine Resistant HBV Target DNA

HBV PCR primers were prepared from ones capable of detecting HBV DNA polymerase gene according to the known method [Oligonucleotide chip for Detection of Lamivudine-resistant Hepatitis B virus, Jang et al., J Clin Microbiol 2004, 4181-4188]. The primers have nucleotide sequences as shown in the following Table 2. SEQ ID Nos. 19 and 20 are outer primers for primary PCR reaction, while SEQ ID Nos. 21 and 22 are inner primers labeled with biotin.

TABLE 2

| | |
|---|---|
| | Size of |
| | PCR |
| Primer nucleotide sequence (5′→3′) | product |

| | | | |
|---|---|---|---|
| BF105 | Sense | 5′-TCCTGCTGCTATGCCTCATC-3′ | 500 bp |
| | (SEQ ID No. 19) | | |
| BF112 | Anti-sense | 5′-TTCCGTCGACATATCCCATGAAGTTAA | |
| | (SEQ ID No. 20) | GGGA-3′ | |
| HB-F3 | Sense | 5′-biotin- | 200 bp |
| | (SEQ ID No. 21) | CTTGTATTCCCATCATCCCATCATC-3′ | |
| HB-R2 | Antisense | 5′-biotin- | |
| | (SEQ ID No. 22) | GAAAAGAAAATTGGTAACAGCGGTA-3′ | |

In order to ensure the emission of fluorescence after the hybridization, the primers were prepared to have 5′-labeled biotin, which binds with Streptavidin-cyanine 5 upon hybridization. The primers were synthesized by Bioneer Corporation in Korea.

### Example 3

### Obtainment of a Large Amount of Recombinant HBV Clone

HBV clonal DNA obtained from GeneIn, Co., Ltd. (Korea) was transformed to E. coli JM109 (Stratagene, USA) to obtain a large amount of the clone. Genotypes of the wild type and mutant type of HBV associated with lamivudine resistance were confirmed by nucleotide sequencing.

### Example 4

### Preparation of Target Nucleic Acid

DNA extracted from clinical samples obtained from GeneIn Co., Ltd. (Korea), and individual wild types and mutant types associated with lamivudine resistance of HBV obtained according to Example 3 were employed. Primary and secondary PCRs were carried out to amplify DNA. The Primary PCR was performed by using BF105 and BF112 primers as shown in Table 1, under the following condition:

For a reaction mixture comprising 3 μl of a template DNA solution (50 ng/μl), 1 μl of sense primer (10 pmol/μl), 1 μl of anti-sense primer (10 pmol/μl), 1 μl of dNTP (10 mM), 5 μl of 10× Taq buffer (containing MgCl₂), 10 μl of BD (Band Doctor™) (Solgent Co., Korea), 0.2 μl of Taq (5 unit/μl, from SolGent Co., Ltd., Korea), and 28.8 μl of distilled water, pretreatment at 94° C. for 4 minutes, and runs of 30 cycles, each cycle consisting of 94° C. for 1 minute, 58° C. for 1 minute and 72° C. for 1 minute.

The Secondary PCR was performed by using HB—F3 and HB—R2 primers as shown in Table 1, under the following condition:

For a reaction mixture comprising 1 μl of the primary PCR reaction solution, 1 μl of sense primer (10 pmol/μl), 0.75 μl of anti-sense primer (10 pmol/μl), 1 μl of dNTP (10 mM), 5 μl of 10× Taq buffer (containing MgCl₂), 10 μl of BD (Band Doctor™) (Solgent Co., Korea), 0.2 μl of Taq (5 unit/μl, from SolGent Co., Ltd., Korea), and 30.8 μl of distilled water, pretreatment at 94° C. for 4 minutes, and runs of 30 cycles, each cycle consisting of 94° C. for 1 minute, 58° C. for 1 minute and 72° C. for 1 minute.

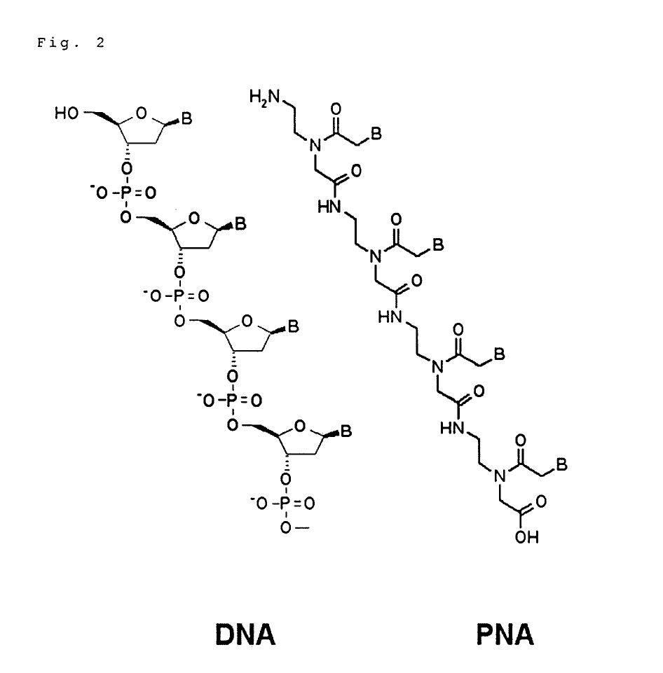

Upon completion of the reaction, to the secondary PCR product (200 bp, 5 μl) was added 1 μl of gel loading buffer (from SunBio, Korea), and the mixture was subjected to electrophoresis on 1.5% agarose gel. After staining with 1 μg/ml of ethidium bromide (EtBr), the product was observed under a UV-transilluminator. The results of electrophoresis are shown in FIG. 3.

### Example 5

### Manufacture of PNA Chips

The purified PNA oligomers of SEQ ID Nos. 1 to 7 and 9 to 18 (in SEQ ID No. 1, N is 3-nitroindole-PNA) as shown in Table 1 were diluted with a spotting buffer to 50 μM. They were spotted on a glass slide functionalized with epoxy group by pin-spotting method, and the slide was allowed to stand at ambient temperature with a constant humidity for 4 hours. It was added to DMF, and washed with ultrasonication for 15 minutes. It was added to DMF supplemented with 0.1 M succinic anhydride, and the unreacted amine group was eliminated at 40° C. for 2 hours. Upon completion of the reaction, the reaction solution was removed, and the slide was washed sequentially with DMF and triple distilled water, with ultrasonication for 15 minutes. Then, 100 mM of Tris-HCl buffer containing 0.1. M of ethanolamine was added thereto to inactivate the residual epoxy groups on the surface of the slide. The glass slide was further washed twice with triple distilled water with ultrasonication for 15 minutes, and the slide was treated with boiling water for 5 minutes, washed with triple distilled water for 5 minutes, and then, dried. Then, a silicon reactor capable of comprising 100 μl of hybridization solution was attached onto the slide. FIG. 4 schematically shows the kinds and positions of the probes on the PNA chip.

### Comparative Example 1

### Manufacture of DNA Chips

According to the known method [Oligonucleotide chip for Detection of Lamivudine-resistant Hepatitis B virus, Jang, et al., J Clin Microbiol 2004, 4181-4188], DNA probes for detecting lamivudine resistant HBV, having an amine group at the N-terminus were mixed with 3×SSC spotting buffer and immobilized on a slide CSS-100 (from Cell, U.S.A.) functionalized with aldehyde group. Then, aldehyde group that had not reacted with amine group was reduced with sodium borohydrate (NaBH₄) solution, and the slide was dried. A silicon reactor capable of comprising 100 μl of hybridization solution was attached onto the glass slide to manufacture a DNA chip.

### Experimental Example 1

### Hybridization with Target Nucleic Acid on the PNA Chip and the DNA Chip

The biotin-labeled PCR product of 5 μl was added to 100 μl of hybridization buffer, and Streptavidin-cy5 was added thereto for emission of fluorescence. Hybridization mixture (100 μl) was injected through the opening of the silicon reactor of the glass slide, each slide prepared from Example 5 and Comparative Example 1, and reaction was performed at 40° C. for 2 hours. Upon completion of the reaction, the reaction mixture was washed with washing buffer twice at ambient temperature for 5 minutes, and then, dried. By using a fluorescent scanner, the glass slide was analyzed to get image (GenePix 4000B, Exon, U.S.A.).

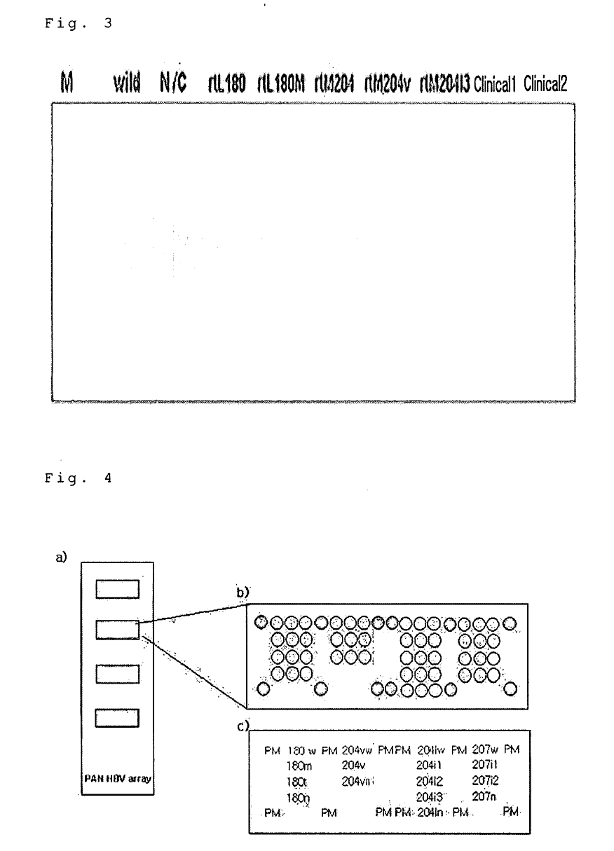

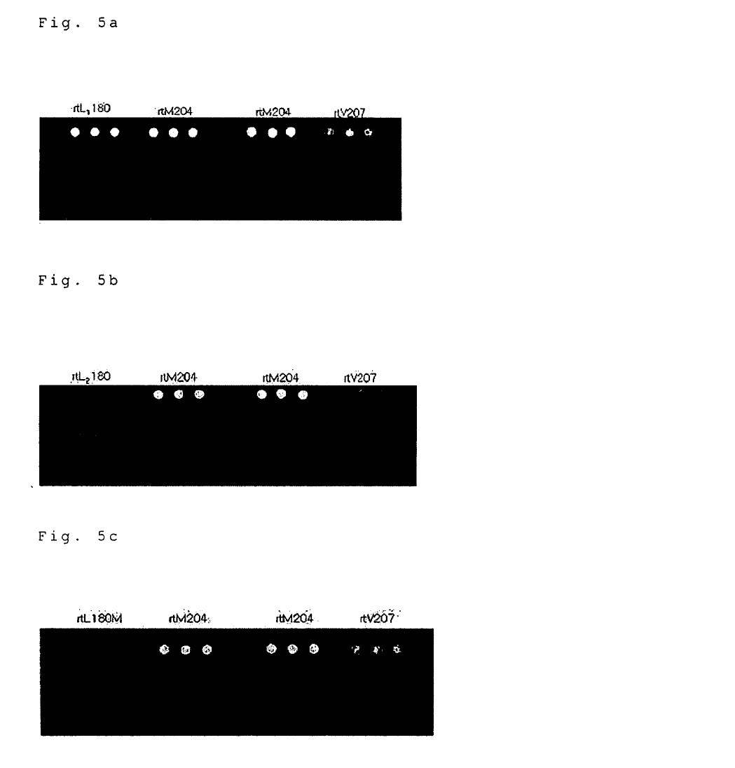

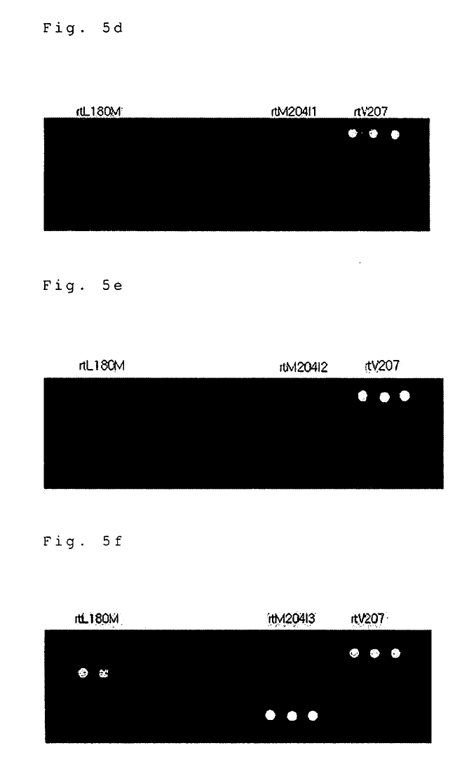

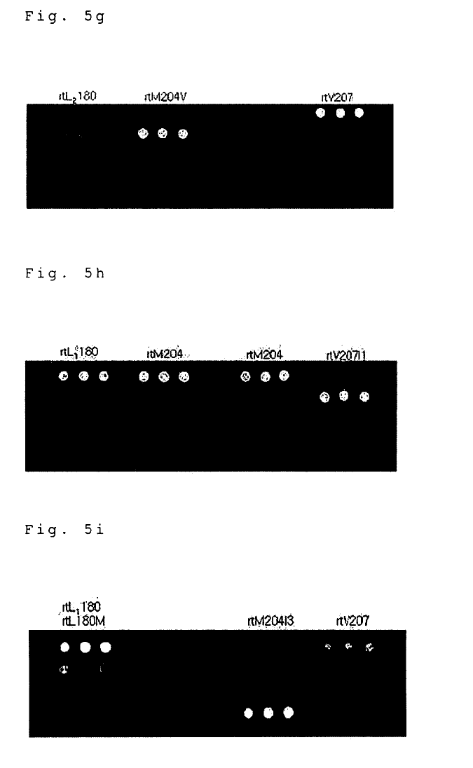

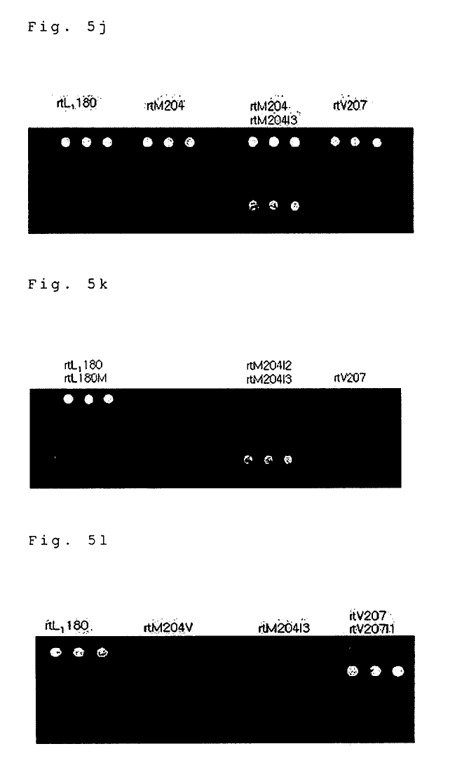

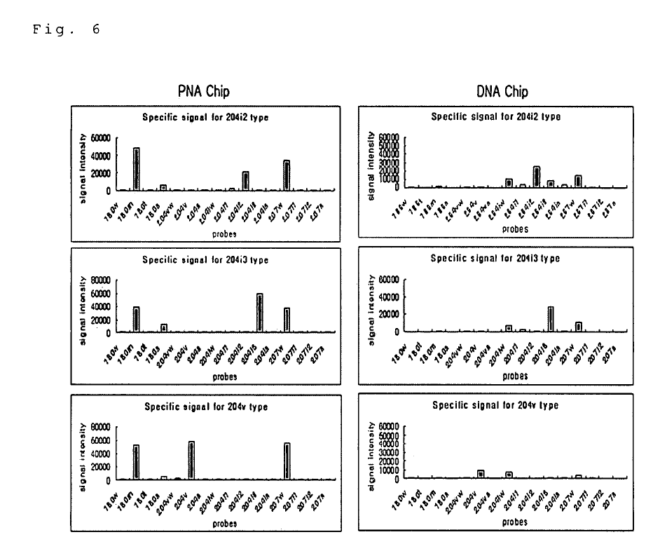

FIGS. 5a to 5l show the results of detection of wild type or lamivudine resistant variants (mutants) of HBV by using the PNA chip (Example 5). FIGS. 5a and 5b show the detected wild type of rtL180, rtM204 and rtV207 of HBV. FIG. 5c shows the detected mutant type of rtL180M wherein leucine at rtL180 was altered to methionine, and wild types rtM204 and rtV207. FIG. 5d shows the detected mutant type of rtM204 i1 wherein rtL180M and rtM204 were altered to isoleucine; FIG. 5e shows the detected mutant type of rtM204i2; FIG. 5f shows the detected mutant type of rtM204i3;

FIG. 5g shows the detected mutant type of rtM204V wherein rtM204 was altered to valine; FIG. 5h shows the detected mutant of rtV207 μl wherein rtV207 was altered to isoleucine; FIG. 5i shows the detected wild type rtL180 and mutant type rtL180M together in clinical sample; FIG. 5j shows the detected wild type rtM204 and mutant type rtM204i3 together in clinical sample; FIG. 5k shows the detected wild type rtL204 and mutant types rtM207 and rtM204i3 together in clinical sample; and FIG. 5l shows the detected mutant type rtM204V, wild type rtV207 and mutant type rtV207 i1 together in clinical sample.

From those Figures, it was confirmed that probes genotype specifically bound with target nucleic acids without nonspecific cross reaction even in the detection of mutations with a single nucleotide sequence variation. The results conform to the nucleotide sequences of mutant types shown in Table 1, and demonstrate high specificity of the PNA chip.

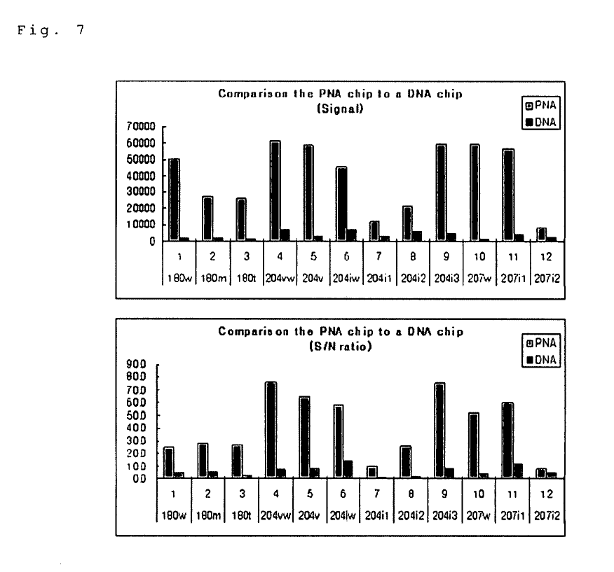

Further, FIG. 6 comparatively shows the quantified detection signals for mutant types of rtM204I2, rtM204I3 and rtM204V, the main sites causing HBV drug resistance, on DNA chip (Comparative Example 1) and PNA chip (Example 5), respectively. FIG. 7 comparatively shows the specific signals and S/N ratios of DNA chip (Comparative Example 1) and PNA chip (Example 5). As can be seen from the Figures, it is confirmed that the PNA chip according to the present invention exhibits much higher specific signal and discrimination as compared to the conventional DNA chip.

According to the present invention, HBV mutants causing lamivudine resistance can be detected and analyzed with excellent sensitivity and specificity in a short time. Thus, the presence of HBV mutants having resistance to lamivudine, a therapeutic agent for chronic hepatitis B, can be rapidly and accurately confirmed, to contribute to effective therapy on chronic hepatitis B, as well as to provide important information on prevention or diagnosis of liver cancer. Further, PNA itself used as probe is very stable against biological enzymes and physical factors, and thus, is not influenced by environments or other factors. Thus, PNA probes are expected to successfully replace DNA probes in commercial diagnosis of HBV infection and antibiotic resistance, for example, in line probe assay, DNA array or the like.

What is claimed is:

1. A PNA (peptide nucleic acid) probe capable of specifically binding with wild type or mutant type at codon 180 in domain B, or codon 204 or 207 in domain C of HBV DNA polymerase gene associated with lamivudine resistance, which consists of the nucleotide sequence as set forth in any one of SEQ ID Nos. 1, 2, 3, 5, 6, 9, 10, 11, 12, 13, 15, 16 and 17.

2. A negative control PNA probe, which hybridizes with neither wild type nor mutant type at codon 180 in domain B, or codon 204 or 207 in domain C of HBV DNA polymerase gene associated with lamivudine resistance, and consists of the nucleotide sequence as set forth in any one of a nucleotide sequence from SEQ ID Nos. 4, 7, 8, 14 and 18.

3. A kit for detecting lamivudine resistant HBV, which comprises one or more PNA probes and a support, the PNA probes being immobilized on the support, wherein the PNA probes are capable of specifically binding with wild type or mutant type at codon 180 in domain B, or codon 204 or 207 in domain C of HBV DNA polymerase gene associated with lamivudine resistance.

4. The kit according to claim 3, wherein the PNA probe consists of the nucleotide sequence as set forth in any one of SEQ ID Nos. 1, 2, 3, 5, 6, 9, 10, 11, 12, 13, 15, 16 and 17.

5. The kit according to claim 3, further comprising one or more negative control probes immobilized on the support, the negative control probes having varied nucleotide sequences from those of the PNA probes so as to hybridize with neither wild type nor mutant type at codon 180 in domain B, or codon 204 or 207 in domain C of HBV DNA polymerase gene to induce lamivudine resistance.

6. The kit according to claim 5, wherein the negative control probe consists of the nucleotide sequence as set forth in any one of SEQ ID Nos. 4, 7, 8, 14 and 18.

7. The kit according to claim 3, wherein the support is selected from the group consisting of glass slide, silica, semiconductor, plastic, gold, silver, magnetic molecule, nylon, polydimethylsiloxane (PDMS), cellulose and nitrocellulose.

8. The kit according to claim 3, wherein the support is in the form of a thin plate, a tube or beads.

9. A method for detecting lamivudine resistant HBV, which comprises the steps of:
(a) adding a reaction sample containing a target DNA to the kit according to claim 3;
(b) subjecting PNA probe(s) in the kit and the target DNA to hybridization; and
(c) detecting the signal from the hybridization of PNA and; DNA.
